(19) United States
(12) Patent Application Publication
(10) Pub. No.: US 2025/0235305 A1
(43) Pub. Date: Jul. 24, 2025

(54) FLOW REGULATION STENT WITH COVERING

(71) Applicant: EDWARDS LIFESCIENCES CORPORATION, Irvine, CA (US)

(72) Inventors: Michael G. Valdez, Riverside, CA (US); Arnold Cruz Tuason, Claremont, CA (US); Tiana (formerly Thu Thi Anh) Tran, Westminster, CA (US)

(73) Assignee: EDWARDS LIFESCIENCES CORPORATION, Irvine, CA (US)

(21) Appl. No.: 19/174,764

(22) Filed: Apr. 9, 2025

Related U.S. Application Data
(63) Continuation of application No. PCT/US2023/076934, filed on Oct. 15, 2023.
(60) Provisional application No. 63/379,277, filed on Oct. 12, 2022.

(51) Int. Cl. A61F 2/07; A61F 2/00; A61F 2/06

(52) U.S. Cl. CPC A61F 2/07; A61F 2002/0081; A61F 2002/068; A61F 2002/075; A61F 2220/0075; A61F 2250/0039

(57) ABSTRACT

A cardiac implant includes a frame forming an inner lumen and sized for delivery into a blood vessel and a covering extending at least partially along the frame to at least partially restrict lateral blood flow from one or more branching blood vessels into the blood vessel. The covering forms one or more folds at or near a distal end of the frame.

### RELATED APPLICATION

This application is a continuation of International Patent Application No. PCT/US2023/076934, filed Oct. 15, 2023, which claims the benefit of U.S. Provisional Patent Application No. 63/379,277, filed on Oct. 12, 2022, the complete disclosures of which are hereby incorporated by reference in their entireties.

### BACKGROUND

The present disclosure generally relates to devices and methods for cardiac blood flow management.

Catheter systems, such as treatment, delivery, and/or deployment catheters, can be used to treat patients internally. For example, delivery catheter systems can be used to deliver and deploy implants at locations inside the body. Implants can be delivered to a treatment site within a patient using transcatheter techniques.

### SUMMARY

Described herein are systems, devices, and methods for managing blood flow within one or more blood vessels of a heart.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

It should be understood that each of the elements disclosed herein can be used with any and all of the elements disclosed herein, even though the specific combination of elements may not be explicitly shown in the figures herein. In other words, based on the explanation of the particular device, one of skill in the art should have little trouble combining the features of certain of two such devices. Therefore, it should be understood that many of the elements are interchangeable, and the invention covers all permutations thereof.

Other objects, features, and advantages of the present invention will become apparent from a consideration of the following detailed description.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes, and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 provides a schematic representation of portions of the splanchnic circulation.

FIG. 2 provides another schematic representation of the splanchnic circulation, illustrating blood flow from the aorta to the IVC.

FIG. 3 illustrates portions of the splanchnic venous circulation acting as a blood reservoir between the aorta and the IVC.

FIG. 4 illustrates blood flow from one or more splanchnic arteries through the splanchnic reservoir, into a hepatic vein and finally into the IVC.

FIG. 5 depicts an example of a system with a delivery catheter for deploying an implant in accordance with one or more examples.

FIGS. 6A-6D illustrate an example blood flow reducing and/or managing stent 601 having a tapered covering in accordance with one or more examples.

FIGS. 7A and 7B illustrate an example blood flow reducing and/or managing stent having a tapered covering in accordance with one or more examples.

FIGS. 8A and 8B illustrate another example stent configured to regulate blood flow within one or more blood vessels in accordance with one or more examples.

FIG. 9 illustrates another example stent configured to regulate blood flow within one or more blood vessels in accordance with one or more examples.

FIG. 10 illustrates another example stent configured to regulate blood flow within one or more blood vessels in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

In humans and other vertebrate animals, blood circulation throughout the body is facilitated by a blood circulatory system comprising various arteries, capillaries, veins, and coronary vessels, which work together with the heart to supply blood to the various regions of the body. The heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The valves may permit fluid flow between the heart and the various arteries of the cardiovascular system.

The following includes a general description of human cardiac anatomy that is relevant to certain inventive features and examples disclosed herein and is included to provide context for certain aspects of the present disclosure.

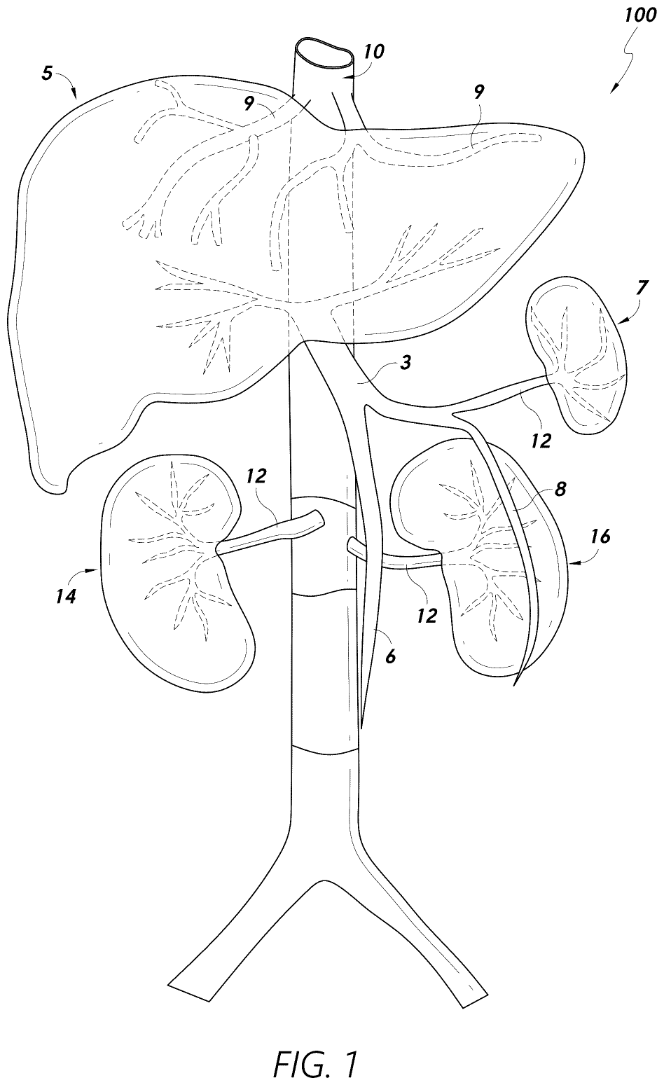

FIG. 1 provides a schematic representation of portions of the splanchnic circulation 100. The term “splanchnic circulation” refers to blood flow originating from the celiac, superior mesenteric, and inferior mesenteric arteries to the abdominal gastrointestinal organs. The splanchnic circulation 100 receives approximately 25% of the cardiac output and holds a similar percentage of the total blood volume under normal conditions. The splanchnic circulation 100 can act as a site of cardiac output regulation and/or as a blood reservoir. Multiple regulatory pathways are involved in the distribution of the splanchnic circulation.

Total flow to the splanchnic viscera is controlled by resistance vessels in the mesenteric and hepatic arterial systems. The venous effluents from the splanchnic viscera converge to form the portal vein 3, which supplies approximately 75% of the total blood supply to the liver 5. The portal blood not only is high in substrate concentrations resulting from intestinal absorption but also tends to contain bacteria and endotoxin.

Renal veins 12 drain blood from the right kidney 14 and left kidney 16 and connect to the inferior vena cava 10 (IVC). The superior mesenteric vein 6 is a major venous tributary of the abdominal cavity that lies laterally to the superior mesenteric artery and serves to drain the vast majority of the organs of the abdominal cavity. The inferior mesenteric vein 8 drains blood from the large intestine. The splenic vein 12 is a blood vessel that drains blood from the spleen, the stomach fundus, and part of the pancreas.

The portal vein 3 receives blood from the stomach, intestines, pancreas, and spleen 7 and carries it into the liver 5 through the porta hepatis. The porta hepatis serves as the point of entry for the portal vein 3 and the proper hepatic artery, and is the point of exit for the bile passages.

Following processing of the blood by the liver 5, the blood collects in the central vein at the core of the lobule. Blood from these central veins ultimately converges in the right and left hepatic veins 9, which exit the superior surface of the liver 5 and empty into the IVC 10 to be distributed to the rest of the body.

The splanchnic venous circulation 100 is highly compliant and can act as a blood reservoir that can be recruited in order to support the need for increased stressed blood volume during periods of elevated sympathetic tone, such as exertion, in order to support increased cardiac output and vasodilation of peripheral vessels supporting active muscles. However, heart failure patients can have multiple comorbidities that prevent them from using that additional blood volume. Such comorbidities can include chronotropic incompetence, inability to increase stroke volume, and/or peripheral microvascular dysfunction. This can lead to venous congestion and/or abrupt rises in pulmonary capillary wedge pressure (PCWP).

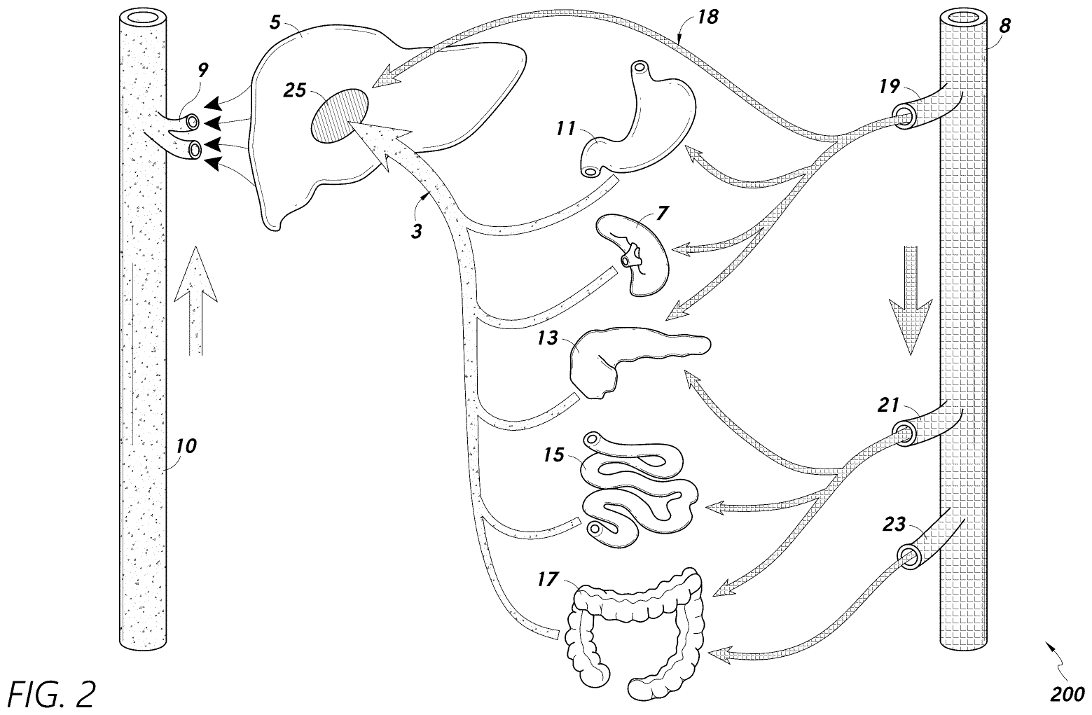

FIG. 2 provides another schematic representation of the splanchnic circulation 200, illustrating blood flow from the aorta 8 to the IVC 10. Blood travels from the aorta 8 to the abdominal gastrointestinal organs including the stomach 11, liver 5, spleen, 7, pancreas 13, small intestine 15, and large intestine 17. The splanchnic circulation 200 comprises three major branches of the abdominal aorta 9, including the coeliac artery 19, the superior mesenteric artery 21 (SMA), and the inferior mesenteric artery 23 (IMA). The hepatic portal circulation (e.g., the hepatic artery 18 and/or portal vein 3) delivers the majority of blood flow to the liver 5.

The coeliac artery 19 is the first major division of the abdominal aorta 8, branching at T12 in a horizontal direction ˜1.25 cm in length. It shows three main divisions such as the left gastric artery, common hepatic artery 18, and splenic artery and is the primary blood supply to the stomach 11, upper duodenum, spleen 7, and pancreas 13.

The SMA 21 arises from the abdominal aorta 8 anteriorly at L1, usually 1 cm inferior to the coeliac artery 19. The five major divisions of the SMA 21 are the inferior pancreaticoduodenal artery, intestinal arteries, ileocolic, right colic, and middle colic arteries. The SMA 21 supplies the lower part of the duodenum, jejunum, ileum, caecum, appendix, ascending colon, and two-thirds of the transverse colon. It is the largest of the splanchnic arterial vessels delivering >10% of the cardiac output and therefore has significant implications for embolic mesenteric ischaemia.

The IMA 23 branches anteriorly from the abdominal aorta 8 at L3, midway between the renal arteries and the iliac bifurcation. The main branches of the IMA 23 are the left colic artery, the sigmoid branches, and the superior rectal artery. It forms a watershed with the middle colic artery and supplies blood to the final third of the transverse colon, descending colon, and upper rectum.

Blood flow is conveyed into the liver 5 via the portal vein 3 into sinusoids 25 of the liver 5. The hepatic veins 9 convey the blood from the liver 5 to the IVC 10.

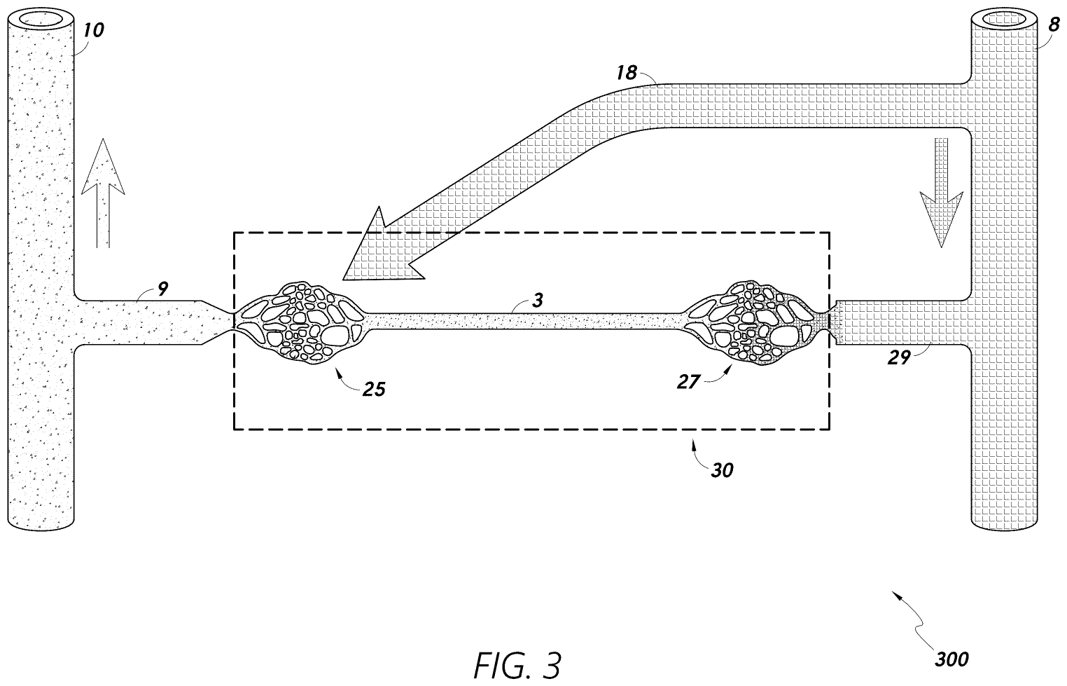

FIG. 3 illustrates portions of the splanchnic venous circulation 300 acting as a blood reservoir 30 between the aorta 8 and the IVC 10. The portal vein 30 conveys blood between the splanchnic organs 27 (e.g., the stomach, spleen, etc.) and the liver sinusoids 25. The liver sinusoids 25 also receive blood from the hepatic artery 18. The splanchnic organs 27 receive blood from the aorta 8 via various splanchnic arteries 29 (e.g., the SMA, IMA, etc.). The amount of blood contained in the portal vein 3 at any given time can be variable.

For some patients (especially patients experiencing heart failure) fluid redistribution from the splanchnic venous reservoir 30 to the IVC 10 and/or stressed blood volume can contribute to increases in central venous pressure (CVP), pulmonary artery pressure, and/or PCWP. This can be especially problematic during periods of elevated sympathetic tone, such as exertion, and/or can lead to pulmonary congestion that can impact a patient's quality of life and/or can lead to acute decompensation.

The splanchnic venous circulation 300, and particularly the portal vein 3, can advantageously provide a blood reserve to support the need for increased stressed blood volume during periods of elevated sympathetic tone. Because blood flow from the splanchnic venous circulation 300 is directed through the hepatic veins 9 and into the IVC 10, devices placed into the hepatic veins 9 and/or IVC 10 to limit blood flow can allow the reservoir 30 to expand with increased blood volume.

Examples described herein can relate to devices and/or methods that can advantageously limit, stagnate, and/or impede blood flow into the IVC 10 from the hepatic veins 9 to increase the pressure gradient between the IVC 10 and the liver and/or splanchnic venous circulation 300. In some examples, one or more flow-regulating implants may be configured for placement at least partially within the hepatic veins 9 and/or IVC 10 and/or at one or more junctions between the hepatic veins 9 and the IVC 10. As a result, blood flowing from the splanchnic venous reservoir 30 into the hepatic veins 9 can be slowed to increase blood volume in the splanchnic venous reservoir 30.

Some approaches to reducing volume redistribution can involve placing fixed orifice flow restrictors at or near the IVC 10. However, while restricting the flow from the hepatic veins 9 can be beneficial in preventing volume redistribution, too much restriction can cause hepatic congestion. It would therefore be advantageous to modulate the response and increase restriction only during volume redistribution.

Some examples presented herein relate to methods and/or devices for reducing and/or restricting blood flow in the splanchnic system and/or from the hepatic veins into the IVC 10. Example devices can include a collapsible stent (e.g., frame) configured for placement within the IVC 10 and/or other blood vessel. Devices can comprise one or more bladders composed of any suitable materials (e.g., tissue, polymer, and/or similar material). The one or more bladders can be attached around an inner and/or outer surface of the stent to at least partially block lateral blood flow from the hepatic veins and/or other branching blood vessels into the blood vessel.

In some examples, the bladder can have a wavy form and/or a distance and/or gap between the stent and the bladder can vary across the surface of the bladder. For example, the bladder may connect to the stent at certain points (e.g., at points where the stent extends inward and/or at posts of the frame) and/or may extend away from the stent at certain points to create a gap between the stent and the bladder. The bladder may have an angled and/or tapered orientation with respect to the stent to create a variable gap between the bladder and the stent along a length of the stent.

The stent may have a variable diameter and/or may have a larger diameter at inflow and/or outflow end portions of the stent than at a midsection of the stent. The bladder may be situated around an inner and/or outer surface of the midsection of the stent to create a gap between the bladder and the outflow orifices of the hepatic veins to allow a reduced amount of blood flow from the hepatic veins up into the right atrium.

The bladder may comprise multiple segments/sections with a gap and/or space between the segments to allow blood flow between the segments. The bladder may be situated around an inner and/or outer surface of the stent. In some examples, the bladder may be configured to at least partially block and/or restrict flow from multiple hepatic veins.

The various implants described herein can be implanted using a transcatheter and/or transvenous approach. Example stents can be compressible during delivery and/or configured to expand following delivery. The stent body can be configured for placement in the IVC and/or other blood vessel. An implant can comprise a bladder (e.g., covering) composed of tissue, polymer, and/or similar materials configured to block and/or impede lateral blood flow from the hepatic veins and/or other branching blood vessels.

In some examples, the bladder may be situated at least partially within the stent and/or frame and/or may form a wavy structure to create gaps between the bladder and the stent. The bladder may have attachment points to the stent at the peaks and/or crests of the wavy structure of the bladder. The bladder may be situated within the stent and/or may have an angled and/or tapered orientation with respect to the stent such that a gap between the bladder and the stent increases towards the outflow (e.g., distal) end portion of the stent.

The stent may have a larger diameter at the end portions than at a midsection of the stent to create a gap between the midsection of the stent and the inflow orifices of the hepatic veins. In some examples, the bladder may be situated at the midsection of the stent. The bladder may be situated along an inner and/or outer surface of the stent. In some examples, the bladder comprises two segments with a separation between the segments to allow blood flow between the segments.

In some examples, a stent and/or similar device may provide blood flow regulation to any blood vessel in or on which it is placed. The term “stent” is used herein in accordance with its broad and ordinary meaning and may refer to any device configured to be implanted in a blood vessel for managing blood flow of the blood vessel.

In some examples, a stent may be deployed within a blood vessel. However, one or more stents may additionally or alternatively be configured to be positioned around an outer surface of the blood vessel. A stent may comprise one or more hooks and/or other attachment mechanisms adapted to help secure the stent to the tissue of the blood vessel wall.

A stent may comprise a stent wall defining an elongated tubular member having a first end with a first opening. The tubular member may further comprise a second end with a second opening, a lumen extending between the first opening and the second opening, and/or a stent length extending between the first end and the second end. The stent wall may comprise an open cell wall and/or may be adapted to be secured to a blood vessel wall of a blood vessel, such as via outward-directed hooks and/or endothelialization. In some examples, one or more stents may be at least partially composed of a shape-memory material, such as Nitinol.

A stent may be configured to be percutaneously delivered to a blood vessel in a compressed configuration. Once within the blood vessel, the stent and/or stent wall of the stent may be configured to be radially expanded into direct surface contact with the blood vessel wall (e.g., the aortic wall of an aorta). In some examples, the stent may be configured to be expanded such that the perimeter of a lumen of the stent may approximate and/or exceed a perimeter of the blood vessel at least prior to expansion of the stent. In some cases, a stent configured to expand to an at least slightly greater perimeter than the native blood vessel may provide improved traction and/or resistance to migration within the blood vessel.

Some systems described herein may include a catheter and/or an implant such as a stent. The catheter may comprise a catheter distal portion, a catheter proximal portion, and/or an elongate catheter body extending from the catheter distal portion to the catheter proximal portion. In some examples, the catheter may be adapted for the catheter distal portion to be percutaneously advanced within a patient's vasculature to a blood vessel. The catheter distal portion may comprise an expandable balloon adapted to radially expand the stent into contact with the blood vessel wall. The catheter distal portion may comprise a retractable sheath adapted to prevent radial expansion of the stent. For example, the catheter distal portion may be configured to prevent the stent from expanding from a first (e.g., compressed) configuration to a second (e.g., expanded) configuration.

Stents may comprise barbs and/or other attachment mechanisms which can prevent migration and/or can help hold the stent securely to a blood vessel wall in order to cause the blood vessel cross-sectional shape to change responsive to the stent shape change. Such barbs and/or other attachment devices may be adapted to engage the wall of the blood vessel in which the stent is deployed.

In some examples, a stent may comprise one or more anchors extending from the main stent body (such as any of the main stent bodies disclosed herein). Each of the one or more anchors may be adapted to be deployed into engagement with tissue of a branch blood vessel or other vascular structure, such as where a branch blood vessel branches away from the main blood vessel. The branch blood vessel may have a diameter that may be smaller than the diameter of the main blood vessel in which the main stent body is deployed. One or more of the anchors may extend from the main stent body at a position between the first end and the second end of the main stent body, or from the first end or from the second end of the main stent body. One or more of the anchors may be adapted to be deployed into contact with walls of a renal artery, or into the walls of an iliac artery. One or more of the anchors may comprise a wireform (e.g., formed from a shape-memory material) and/or may be adapted to pass within the branch blood vessel and/or adapted to engage the wall tissue of the branch blood vessel such as an iliac or renal artery. One or more of the anchors may comprise an anchor stent body with an anchor stent wall defining an anchor stent lumen, and/or the anchor stent body may be adapted to be radially expanded into contact with a wall of a branch blood vessel such as an iliac or renal artery. The anchor stent body may be at least partially composed of a shape-memory material and/or may be biased toward a configuration wherein the anchor stent lumen comprises a cross-sectional shape which is non-circular (e.g., an oval, triangle, peanut, or kidney shape). In some examples, the anchor stent body may have an overall length in the range from 0.5 and 7 cm.

The systems, devices, and/or methods described herein can be utilized in various catheter-based procedures, including minimally invasive procedures and percutaneous procedures. In some examples the methods/systems/devices may involve trans-aortic deliveries through a small chest (or abdominal) incision. In other examples, the methods/systems/devices can be used in minimally invasive surgical procedures. In yet other examples, the methods/systems/devices can be used in percutaneous procedures, such as via a catheter or catheters into the patient's arterial system (e.g., through the femoral or brachial arteries).

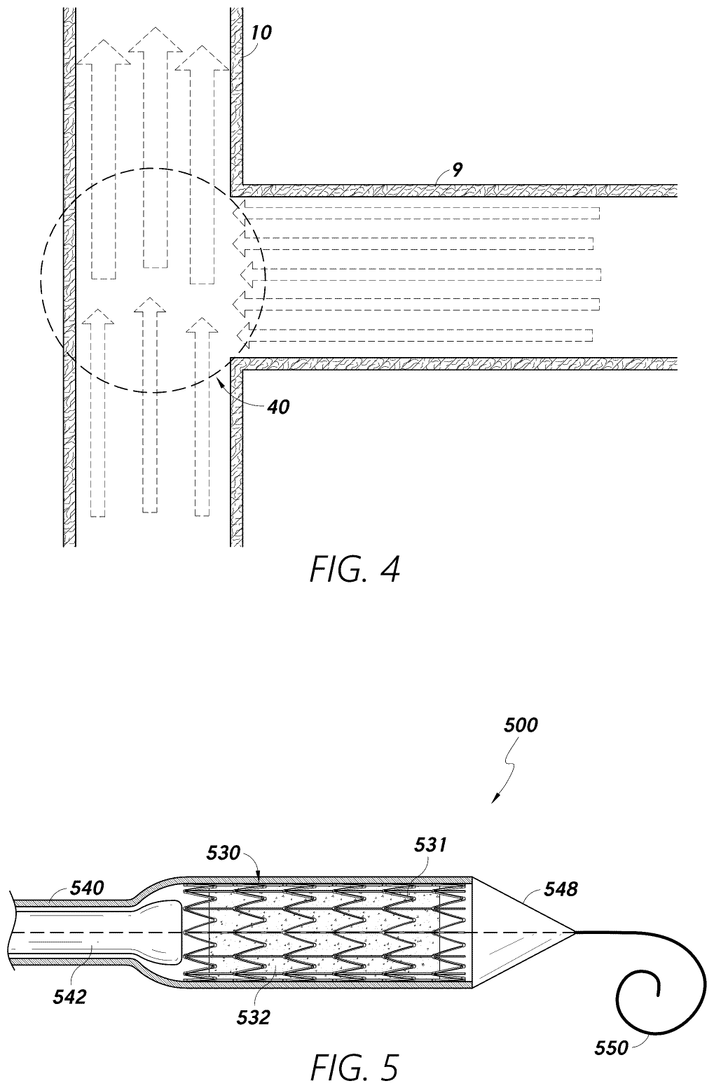

FIG. 4 illustrates blood flow from one or more splanchnic arteries through the splanchnic reservoir, into a hepatic vein 9 and finally into the IVC 10. While only a single hepatic vein 9 is shown in FIG. 4 for illustrative purposes, multiple hepatic veins may convey blood from the reservoir into the IVC 10. The hepatic vein 9 may feed into a junction portion 40 of the IVC 10. Accordingly, to limit blood flow into the IVC 10, one or more flow-limiting implants may be configured for placement within the hepatic vein 9 and/or at least partially within the junction portion 40 of the IVC 10.

The present disclosure provides methods and devices (including various medical implants) for managing blood flow within a human body. The term “implant” is used herein according to its plain and/ordinary meaning and may refer to any medical implant, frame, valve, shunt, stent, anchor, and/or similar devices for use in treating various conditions in a human body. Implants may be delivered percutaneously and/or via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an IVC and/or hepatic vein, including for example delivery catheters and/or cannulas.

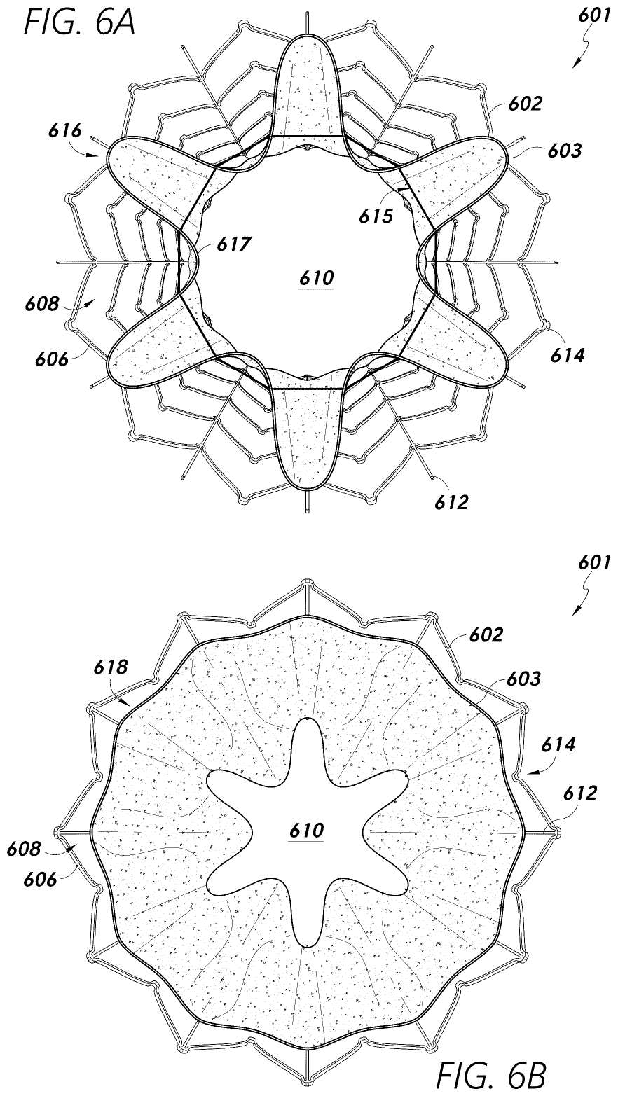

FIG. 5 depicts an example of a system 500 with a delivery catheter 540 (e.g., sheath and/or shaft) for deploying an implant 530 in accordance with one or more examples. The delivery catheter 540 may comprise a proximal end with a handle and/or controls thereon. The delivery catheter 540 may further comprise a distal end 548 adapted to be advanced into a blood vessel, such as via percutaneous methods. The distal end 548 may have a conical and/or pointed shape and/or may be configured to dilate one or more blood vessels and/or openings during delivery through a body.

The delivery catheter 540 may be configured to cover and/or protect the implant 530 during delivery. In some examples, the delivery catheter 540 may be configured to be retracted to permit the implant 530 to expand at a desired deployment position. The delivery catheter 540 may comprise an expandable balloon which can be selectively expanded in order to expand the implant 530 firmly into contact with a blood vessel wall. For a self-expanding implant 530, the balloon can add further expansion to firmly engage the implant 530 against a blood vessel wall. In some examples, components of the system 500 may be configured to be loaded onto the delivery catheter 540 via various methods. For example, radial crimping, folding, and/or rolling may be used to load onto the catheter 540. The implant 530 can comprise a frame 531 and/or a covering 532. In some examples, a pusher 542 may be extended within the catheter 540 to press the implant 530 towards the distal end 548 and/or out of the catheter 540.

The distal end 548 of the delivery catheter 540 may be advanced into a blood vessel over a guidewire 550 to a desired deployment location. The delivery catheter 540 may be retracted, at which point a self-expanding implant 530 may be configured to radially expand. After the implant 530 is properly deployed, the catheter 540 can be withdrawn from the patient. The implant 530 may form an inner lumen having a cylindrical and/or other shape. The frame 531 of the implant 530 may have a generally cylindrical and/or tubular form.

Various approaches for treatments are within the scope of this disclosure. In some examples, artery access may be obtained via an access sheath dimensioned for use in some procedures. An incision may be created in a patient, leading to an internal blood vessel (e.g., a femoral artery). The distal end of the access sheath may be advanced through the incision and internal blood and into a desired position within the target blood vessel, with the catheter handle positioned outside the patient adjacent the incision/access site. Echo and/or fluoroscopic and/or other visualization techniques may be used to confirm proper position of the implant 530. The treatment and/or implant deployment can occur, such as by deploying the implant 530 at the target location. Once the proper deployment is confirmed, the catheter 540 can be removed from the patient, and the incision(s) may be closed, for example via sutures.

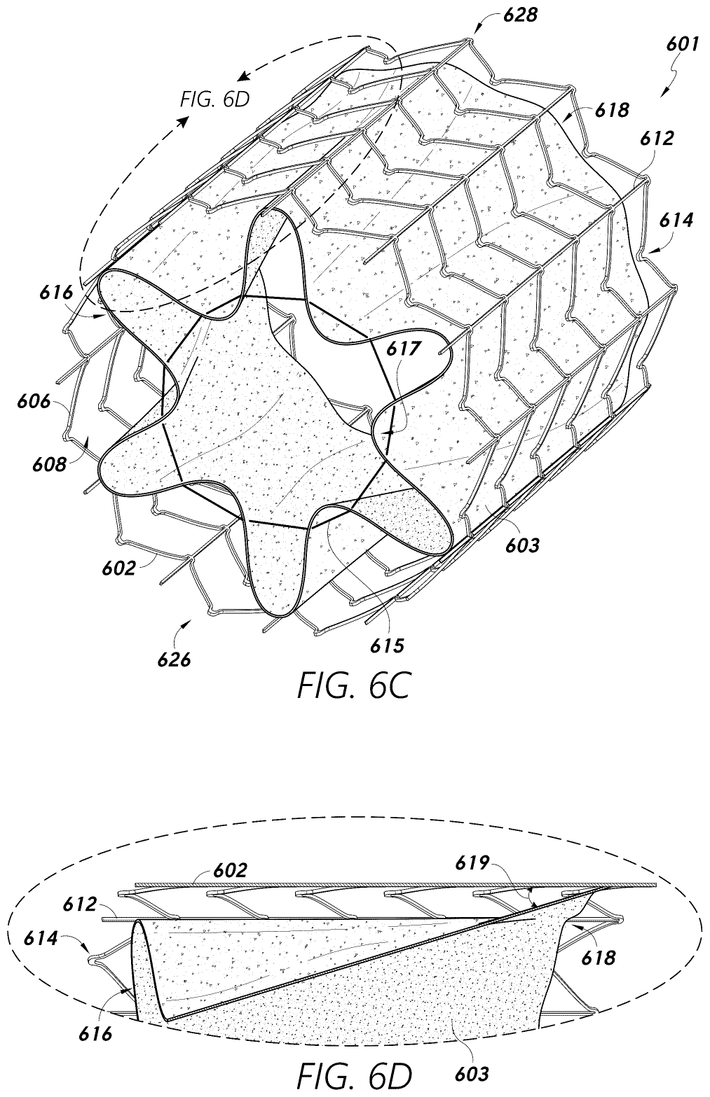

FIGS. 6A-6D illustrate an example blood flow reducing and/or managing stent 601 having a tapered covering 603 in accordance with one or more examples. The stent 601 can comprise a frame 602 and/or a covering 603 (e.g., bladder, skirt, etc.) extending at least partially along an inner and/or outer surface of the frame 602. FIG. 6A provides an overhead view of the implant 601. FIG. 6B provides an underside view of the implant 601. FIG. 6C provides a perspective view of the implant 601. FIG. 6D provides a cross-sectional view of a portion of the implant 601.

The frame 602 may be at least partially collapsible and/or expandable. For example, the frame 602 may be configured to assume a compressed form within a catheter during delivery to a target location within a body (e.g., into an IVC). In some examples, the frame 602 may comprise a network of wires and/or struts 606 forming one or cells 608 and/or openings through the frame 602. The cells 608 may have any suitable size and/or shape. While the cells 608 are shown having a triangular bracket and/or angle bracket form (e.g., forming an acute angle), the cells 608 may have other shapes, including rectangle and/or oval shapes.

The struts 606 may be configured to bend and/or navigate with respect to each other to provide flexibility to the stent 601. For example, the struts 606 may be configured to bend to allow the stent 601 to assume a generally compressed form within a catheter and/or other delivery device. Upon removal from the catheter and/or other delivery device, the stent 601 and/or struts 606 may be configured to relax and/or assume a default expanded and/or relaxed form.

In some examples, the frame 602 may have a variable and/or inconsistent diameter along a circumference of the frame 602, as shown in FIG. 6A. For example, the struts 606 of the frame 602 may be configured to alternatively extend at least partially inwardly (e.g., towards an inner lumen 610 of the stent 601) and/or outwardly (e.g., away from the inner lumen 610 of the stent 601). The frame 602 may comprise inner posts 612 configured to form a first diameter of the stent 601 and/or the one or more struts 606 may be configured to extend away from the inner lumen 610 between the inner posts 612. A “post” may include any generally straight and/or elongate struts 606, wires, bars, arms, cords, and/or similar devices and/or may be configured to form and/or maintain a desired structure (e.g., a tubular and/or cylindrical structure) of the stent 601. In some examples, the struts 606 may be configured to form pointed and/or angled ends 614 between the inner posts 612 and/or the pointed and/or angled ends 614 may form a second diameter of the stent 601 that is greater than the first diameter of the stent 601. The inner posts 612 may be configured to extend along a length of the stent 601 and/or may be configured to attach to the covering 603 at various points.

The one or more posts 612 may be configured to extend generally linearly along a length of the stent 601. The one or more struts 606 may connect to the posts 612 and/or may interconnect the posts 612. In some examples, the struts 606 may extend at various angles from the posts 612 and/or may comprise bends and/or changes in direction between the posts 612. For examples, the one or more struts 606 may have zig-zag forms and/or may have approximately 45-degree bends between adjacent posts 612. The struts 606 and/or posts 612 may extend around and/or form a tubular and/or cylindrical frame around an inner lumen 610.

While the covering 603 is shown extending along and/or attached to an inner surface of the frame 602, the covering 603 may alternatively extend along an outer surface of the stent 601 and/or the stent 601 may comprise an additional covering 603 extending along at least a portion of the stent 601. The covering 603 may be at least partially composed of tissue, cloth, polymer, and/or other similar materials and/or may have a generally flexible and/or soft structure to allow bending and/or folding of the covering 603.

In some examples, the covering 603 may be configured to attach (e.g., connect, couple, adhere) to one or more inner posts 612 of the frame 602. The covering 603 may form any number of attachments with the frame 602. In some examples, the covering 603 may be configured to attach to alternating inner posts 612 of the frame 602. For example, a first end 616 of the covering 603 may be configured to every other inner post 612 (e.g., approximately half of the inner posts 612) at or near a first end 626 of the frame 602 (e.g., an outflow end of the stent 601). The covering 603 may be configured to form folds 617 and/or pleats between attachment points of the first end 616 of the covering 603 and the inner posts 612. The folds 617 may form inner peaks approximately in-line with inner posts 612 that the first end 616 of the covering 603 is not attached to. The first end 616 of the covering 603 may have a generally star-shaped form in which the folds 617 form a smaller diameter than the inner posts 612 of the frame 602.

A second end 618 of the covering 603 may be configured to attach to every inner post 612 of the frame 602 at or near a second end 628 of the frame 602 (e.g., an inflow end of the stent 601). For example, the second end 618 of the covering 603 may have a generally circular form to approximate a generally circular first diameter formed by the inner struts 606. Accordingly, at least some portions of the covering 603 may have a changing and/or variable diameter between the first end 616 and the second end 618 of the covering 603. For example, the folds 617 of the covering 603 may gradually be pulled closer to the frame 602 along a length of the frame 602 until the folds 617 attach to the frame 602 at the second end 618 of the covering 603. Accordingly, the covering 603 may have a generally tapered form between the folds 617 of the covering 603 at the first end 616 of the covering 603 and the second end 618 of the covering 603. An amount of attachments between the covering 603 and frame 602 at the first end 616 may be approximately half an amount of attachments between the covering 603 and the frame 602 at the second end 618.

The stent 601 may comprise a generally collapsible frame 602 interconnected with a tapered bladder and/or covering 603. The covering 603 may be configured to taper away from an inner diameter of the frame 602 (e.g., away from inner posts 612 of the frame 602). The tapered form of the covering 603 may create a tapered gap between the covering 603 and the frame 602 to regulate blood flow output along and/or through the stent 601. In some examples, the tapered form of the covering 603 can allow the stent 601 to provide variable flow reduction based on placement location of the stent 601 and/or covering 603. For example, the stent 601 may be configured for placement within an IVC and/or at or near one or more inflow junctions of one or more hepatic veins and/or other branching blood vessels. However, the stent 601 may be applied for various blood flow reduction applications.

In some examples, the stent 601 may comprise one or more radiopaque markers (e.g., tantalum markers) configured to facilitate delivery, placement, and/or use of the stent 601. The one or more markers may be attached to the frame 602 and/or covering 603 of the stent 601. For example, one or more markers may be situated at the frame 602 at or near the second end 618 of the covering 603 to facilitate placement of the stent 601 such that the second end 618 of the covering 603 is approximately in-line with a lowest point of a branching blood vessel.

The covering 603 may be configured to be responsive to blood flow from one or more branching blood vessels. For example, when blood from hepatic veins presses laterally against the covering 603, the covering 603 may be configured to be pressed into a generally tapered form away from the hepatic veins. Similarly, blood flow through the lumen 610 of the stent 601 may be configured to press the covering 603 against the frame 602. For example, blood flow through the IVC may press against the folds 617 and/or flatten the folds 617 against the frame 602.

In some examples, the covering 603 may be at least partially adjustable while within the body and/or prior to delivery. For example, the stent 601 may comprise one or more cords 615 (e.g., strings, drawstrings, wires, sutures, etc.) and/or similar devices configured to cause an adjustment of the covering 603. A cord 615 may be configured to extend at least partially through the covering 603 and/or through one or more folds 617 of the covering 603. The cord 615 may be tightened, cinched, and/or tensioned to apply pulling force to the folds 617 and/or covering 603 to increase a tapering angle 619 of the covering 603 and/or to pull the covering 603 away from the frame 602. In some examples, one or more cords 615 may extend through the folds 617 at or near the first end 616 of the covering 603. As the one or more cords 615 are tightened and/or cinched, the one or more cords 615 may assume smaller width and/or diameter and/or may pull the folds 617 inwardly.

The covering 603 may be configured to form multiple folds 617 and/or a uniform series of folds 617 around a circumference of the covering 603. Alternatively, the covering 603 may only comprise folds 617 on one side.

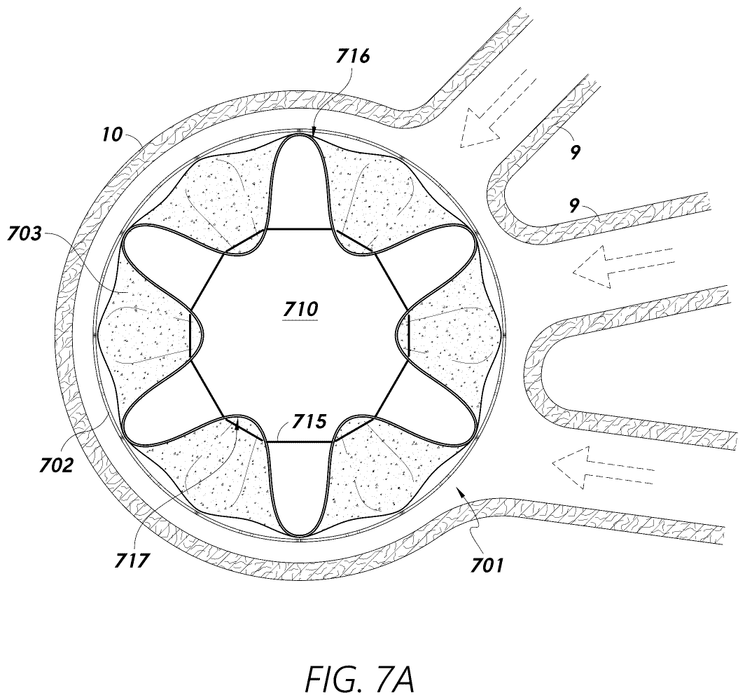

FIGS. 7A and 7B illustrate an example blood flow reducing and/or managing stent 701 having a tapered covering 703 in accordance with one or more examples. The stent 701 can comprise a frame 702 and/or a covering 703 (e.g., bladder, skirt, etc.) extending at least partially along an inner and/or outer surface of the frame 702. FIG. 7A provides an overhead view of the implant disposed within a blood vessel 10. FIG. 7B provides a perspective side view of the implant in the blood vessel 10.

The frame 702 may be at least partially collapsible and/or expandable. For example, the frame 702 may be configured to assume a compressed form within a catheter during delivery to a target location within a body (e.g., into an IVC). In some examples, the frame 702 may comprise a network of wires and/or struts 706 forming one or cells 708 and/or openings through the frame 702. The cells 708 may have any suitable size and/or shape. While the cells 708 are shown having a triangular bracket and/or angle bracket form (e.g., forming an acute angle), the cells 708 may have other shapes, including rectangle and/or oval shapes.

The struts 706 may be configured to bend and/or navigate with respect to each other to provide flexibility to the stent 701. For example, the struts 706 may be configured to bend to allow the stent 701 to assume a generally compressed form within a catheter and/or other delivery device. Upon removal from the catheter and/or other delivery device, the stent 701 and/or struts 706 may be configured to relax and/or assume a default expanded and/or relaxed form.

In some examples, the frame 702 may have a variable and/or inconsistent diameter along a circumference of the frame 702, as shown in FIG. 7A. For example, the struts 706 of the frame 702 may be configured to alternatively extend at least partially inwardly (e.g., towards an inner lumen 710 of the stent 701) and/or outwardly (e.g., away from the inner lumen 710 of the stent 701). The frame 702 may comprise inner posts 712 configured to form a first diameter of the stent 701 and/or the one or more struts 706 may be configured to extend away from the inner lumen 710 between the inner posts 712. In some examples, the struts 706 may be configured to form pointed and/or angled ends 714 between the inner posts 712 and/or the pointed and/or angled ends 714 may form a second diameter of the stent 701 that is greater than the first diameter of the stent 701. The inner posts 712 may be configured to extend along a length of the stent 701 and/or may be configured to attach to the covering 703 at various points.

While the covering 703 is shown extending along and/or attached to an inner surface of the frame 702, the covering 703 may alternatively extend along an outer surface of the stent 701 and/or the stent 701 may comprise an additional covering 703 extending along at least a portion of the stent 701. The covering 703 may be at least partially composed of tissue, cloth, polymer, and/or other similar materials and/or may have a generally flexible and/or soft structure to allow bending and/or folding of the covering 703.

In some examples, the covering 703 may be configured to attach (e.g., connect, couple, adhere) to one or more inner posts 712 of the frame 702. The covering 703 may be configured to attach to alternating inner posts 712 of the frame 702. For example, a first end 716 of the covering 703 may be configured to every other inner post 712 (e.g., approximately half of the inner posts 712) at or near a first end 726 of the frame 702 (e.g., an outflow end of the stent 701). The covering 703 may be configured to form folds 717 and/or pleats between attachment points of the first end 716 of the covering 703 and the inner posts 712. The folds 717 may form inner peaks approximately in-line with inner posts 712 that the first end 716 of the covering 703 is not attached to. The first end 716 of the covering 703 may have a generally star-shaped form in which the folds 717 form a smaller diameter than the inner posts 712 of the frame 702.

A second end 718 of the covering 703 may be configured to attach to every inner post 712 of the frame 702 at or near a second end 728 of the frame 702 (e.g., an inflow end of the stent 701). For example, the second end 718 of the covering 703 may have a generally circular form to approximate a generally circular first diameter formed by the inner posts 712 and/or angled ends of the struts 706. Accordingly, at least some portions of the covering 703 may have a changing and/or variable diameter between the first end 716 and the second end 718 of the covering 703. For example, the folds 717 of the covering 703 may gradually be pulled closer to the frame 702 along a length of the frame 702 until the folds 717 attach to the frame 702 at the second end 718 of the covering 703. Accordingly, the covering 703 may have a generally tapered form between the folds 717 of the covering 703 at the first end 716 of the covering 703 and the second end 718 of the covering 703.

The stent 701 may comprise a generally collapsible frame 702 interconnected with a tapered bladder and/or covering 703. The covering 703 may be configured to taper away from an inner diameter of the frame 702 (e.g., away from inner posts 712 of the frame 702). The tapered form of the covering 703 may create a tapered gap between the covering 703 and the frame 702 to regulate blood flow output along and/or through the stent 701. In some examples, the tapered form of the covering 703 can allow the stent 701 to provide variable flow reduction based on placement location of the stent 701 and/or covering 703. For example, the stent 701 may be configured for placement within an IVC and/or at or near one or more inflow junctions of one or more hepatic veins and/or other branching blood vessels. However, the stent 701 may be applied for various blood flow reduction applications.

In some examples, the stent 701 may comprise one or more radiopaque markers (e.g., tantalum markers) configured to facilitate delivery, placement, and/or use of the stent 701. The one or more markers may be attached to the frame 702 and/or covering 703 of the stent 701. For example, one or more markers may be situated at the frame 702 at or near the second end 718 of the covering 703 to facilitate placement of the stent 701 such that the second end 718 of the covering 703 is approximately in-line with a lowest point of a branching blood vessel.

The covering 703 may be configured to be responsive to blood flow from one or more branching blood vessels. For example, when blood from hepatic veins presses laterally against the covering 703, the covering 703 may be configured to be pressed into a generally tapered form away from the hepatic veins. Similarly, blood flow through the lumen 710 of the stent 701 may be configured to press the covering 703 against the frame 702. For example, blood flow through the IVC may press against the folds 717 and/or flatten the folds 717 against the frame 702.

In some examples, the covering 703 may be at least partially adjustable while within the body and/or prior to delivery. For example, the stent 701 may comprise one or more cords 715 (e.g., strings, drawstrings, wires, sutures, etc.) and/or similar devices configured to cause an adjustment of the covering 703. A cord 715 may be configured to extend at least partially through the covering 703 and/or through one or more folds 717 of the covering 703. The cord 715 may be tightened and/or tensioned to apply pulling force to the folds 717 and/or covering 703 to increase a tapering angle of the covering 703 and/or to pull the covering 703 away from the frame 702.

The covering 703 may be configured to form multiple folds 717 and/or a uniform series of folds 717 around a circumference of the covering 703. Alternatively, the covering 703 may only comprise folds 717 on one side.

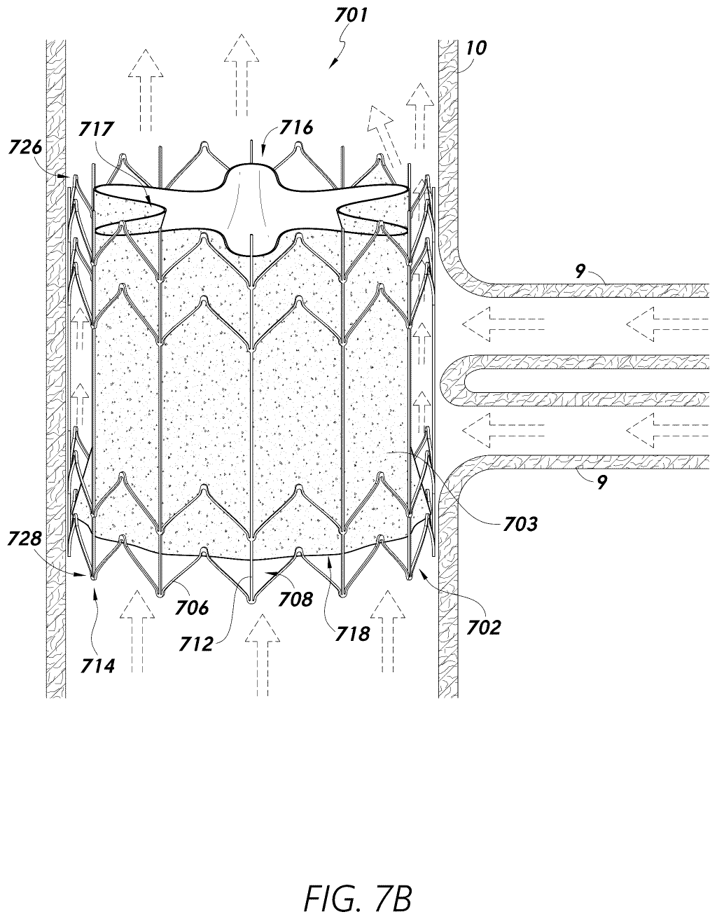

FIGS. 8A and 8B illustrate another example stent 801 configured to regulate blood flow within one or more blood vessels in accordance with one or more examples. The stent 801 may comprise a generally collapsible frame 802 attached to and/or enclosed by a covering 803 composed of tissue, polymer, and/or similar materials. FIG. 8A provides a side view of the stent 801. FIG. 8B provides a side view of the stent 801 disposed within a blood vessel 10.

In some examples, the covering 803 may be attached along an inner diameter and/or around an outer diameter of the frame 802. The frame 802 may comprise a network and/or mesh of struts 806 extending around and/or forming one or more cells 808 (e.g., diamond-shaped and/or other shaped openings through the frame 802).

The frame 802 may have a variable diameter. For example, the frame 802 may comprise a midsection 833 disposed between a proximal section 832 and/or a distal section 834. A diameter and/or width of the midsection 833 may be less than diameters and/or widths of the proximal section 832 and/or distal section 834. In some examples, the stent 801 may be configured for placement such that the midsection 833 may be disposed adjacent to one or more branching blood vessels (e.g., hepatic veins). Accordingly, the frame 802 may be at least partially displaced from the branching blood vessels to allow blood flow from the blood vessels to escape into the IVC and/or other blood vessel in which the stent 801 is disposed.

The midsection 833 may have any suitable diameter. The diameter of the midsection 833 may be determinative of an amount of blood flow allowed from the branching blood vessels. For example, if the diameter of the midsection 833 is relatively small, the frame 802 may allow a relatively high amount of blood flow from the branching blood vessels. Similarly, if the diameter of the midsection 833 is relatively large, the frame 802 may allow a relatively low amount of blood flow from the branching blood vessels.

In some examples, the diameter of the midsection 833 may be at least partially adjustable prior to and/or following delivery of the stent 801. For example, the stent 801 may comprise one or more cords (e.g., sutures) and/or similar devices configured to function like a drawstring and/or similar function to cause a reduction and/or increase of the diameter of the midsection 833. In some examples, one or more cords may be wound through the covering 803 (e.g., through folds of the covering 803) and/or through struts 806 of the frame 802 at or near the midsection 833 and/or may be configured to be tightened and/or tensioned to cause a reduction in diameter of at least a portion of the covering 803 and/or midsection 833. The covering 803 may be configured to form a seal around and/or within the frame 802 to prevent blood flow from the branching blood vessels into a lumen formed by the stent 801 and/or covering 803.

FIG. 8B illustrates the stent 801 disposed within an IVC 10 to regulate blood flow from one or more hepatic veins 9. However, the stent 801 may be configured for use in other application and/or at other anatomies.

In some examples, the covering 803 may comprise one or more holes 811 (e.g., apertures, openings, bypass openings, etc.) configured to allow some blood flow through the covering 803 and/or into the lumen formed by the covering 803 and/or stent 801. The one or more holes 811 may be disposed at any portion(s) of the covering 803 and/or may be disposed at a portion of the covering 803 extending along the midsection 833 of the stent 801. In some examples, the holes 811 may have variable shapes and/or sizes. Sizes and/or shapes of the holes 811 may be determined based on a desired amount of blood flow reduction of the stent 801. In some examples, the positioning and/or size of the holes 811 may be adjusted prior to and/or following delivery of the stent 801. For example, the covering 803 may be flattened to cause the one or more holes 811 to be exposed to the blood flow to increase an amount of blood flow through the covering 803. The one or more holes 811 may be oriented generally perpendicularly to the branching blood vessels and/or may be configured to allow blood flow from the branching blood vessels directly into the lumen.

The covering 803 may be configured to extend at least partially along the proximal section 832 and/or distal section 834 of the frame 802. In the example shown in FIGS. 8A and 8B, the covering 803 extends along the proximal section 832 but not the distal section 834. However, the covering 803 may extend along the distal section 834 and/or may not extend along the proximal section 832. In some examples, the covering 803 at the proximal section 832 may be configured to prevent blood flow from branching blood vessels (e.g., hepatic veins 9) downwards and/or upstream of blood flow within the main blood vessel (e.g., IVC 10).

The stent 801 may be at least partially balloon expandable and/or may be adjustable. For example, the stent 801 may be expanded to a desired size for a particular application. The stent 801 may be configured to maintain multiple desired forms.

In some examples, the proximal section 832 and/or distal section 834 may have increased diameters to facilitate anchoring of the stent 801 within a blood vessel. Different portions of the frame 802 may be expanded at different points to facilitate anchoring of the stent 801. For example, the distal section 834 may be expanded first to anchor the distal section 834 and the proximal section 832 may be expanded later to anchor the proximal section 832 or vice versa.

The frame 802 may comprise a first end 816 (e.g., distal end) and/or a second end 818 (e.g., proximal end). The first end 816 and/or second end 818 may be at least partially flared and/or may increase in diameter relative to other portions of the frame 802.

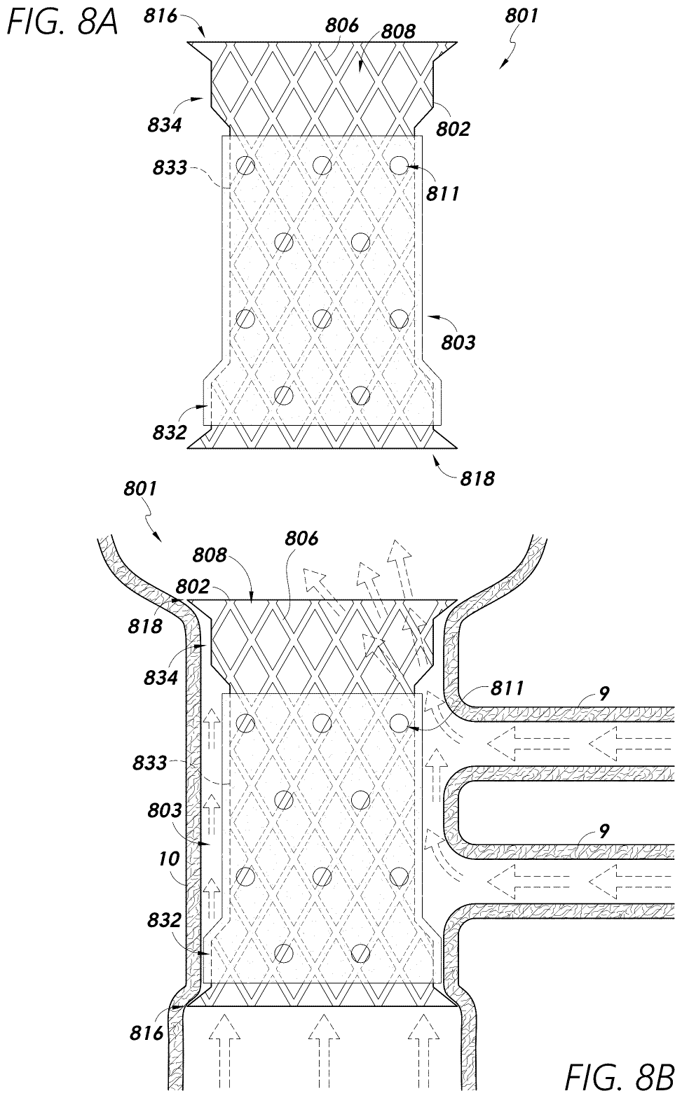

FIG. 9 illustrates another example stent 901 configured to regulate blood flow within one or more blood vessels in accordance with one or more examples. The stent 901 may comprise a generally collapsible frame 902 attached to and/or enclosed by a covering 903 composed of tissue, polymer, and/or similar materials. In some examples, the covering 903 may be attached along an inner diameter and/or around an outer diameter of the frame 902.

The frame 902 may have a variable diameter. For example, the frame 902 may comprise a midsection 933 disposed between a proximal section 932 and/or a distal section 934. A diameter of the midsection 933 may be less than diameters of the proximal section 932 and/or distal section 934. In some examples, the stent 901 may be configured for placement such that the midsection 933 may be disposed adjacent to one or more branching blood vessels (e.g., hepatic veins). Accordingly, the frame 902 may be at least partially displaced from the branching blood vessels to allow blood flow from the blood vessels to escape into the IVC and/or other blood vessel in which the stent 901 is disposed.

The midsection 933 may have any suitable diameter. The diameter of the midsection 933 may be determinative of an amount of blood flow allowed from the branching blood vessels. For example, if the diameter of the midsection 933 is relatively small, the frame 902 may allow a relatively high amount of blood flow from the branching blood vessels. Similarly, if the diameter of the midsection 933 is relatively large, the frame 902 may allow a relatively low amount of blood flow from the branching blood vessels.

In some examples, the diameter of the midsection 933 may be at least partially adjustable prior to and/or following delivery of the stent 901. For example, the stent 901 may comprise one or more cords (e.g., sutures) and/or similar devices configured to function like a drawstring and/or similar function to cause a reduction and/or increase of the diameter of the midsection 933. In some examples, one or more cords may be wound through the covering 903 (e.g., through folds of the covering 903) and/or through struts 906 of the frame 902 at or near the midsection 933 and/or may be configured to be tightened and/or tensioned to cause a reduction in diameter of at least a portion of the covering 903 and/or midsection 933. The covering 903 may be configured to form a seal around and/or within the frame 902 to prevent blood flow from the branching blood vessels into a lumen formed by the stent 901 and/or covering 903.

The stent 901 is shown disposed within an IVC 10 to regulate blood flow from one or more hepatic veins 9. However, the stent 901 may be configured for use in other application and/or at other anatomies.

In some examples, the covering 903 may comprise one or more holes (e.g., apertures, openings, bypass openings, etc.) configured to allow some blood flow through the covering 903 and/or into the lumen formed by the covering 903 and/or stent 901. The one or more holes may be disposed at any portion(s) of the covering 903 and/or may be disposed at a portion of the covering 903 extending along the midsection 933 of the stent 901. In some examples, the holes may have variable shapes and/or sizes. Sizes and/or shapes of the holes may be determined based on a desired amount of blood flow reduction of the stent 901. In some examples, the positioning and/or size of the holes may be adjusted prior to and/or following delivery of the stent 901. For example, the covering 903 may be flattened to cause the one or more holes to be exposed to the blood flow to increase an amount of blood flow through the covering 903. The one or more holes may be oriented generally perpendicularly to the branching blood vessels and/or may be configured to allow blood flow from the branching blood vessels directly into the lumen.

The covering 903 may be configured to extend at least partially along the proximal section 932 and/or distal section 934 of the frame 902. In the example shown in FIG. 9, the covering 903 does not extend along the proximal section 932 or the distal section 934. However, the covering 903 may extend along the distal section 934 and/or the proximal section 932.

The stent 901 may be at least partially balloon expandable and/or may be adjustable. For example, the stent 901 may be expanded to a desired size for a particular application. The stent 901 may be configured to maintain multiple desired forms.

In some examples, the proximal section 932 and/or distal section 934 may have increased diameters to facilitate anchoring of the stent 901 within a blood vessel. Different portions of the frame 902 may be expanded at different points to facilitate anchoring of the stent 901. For example, the distal section 934 may be expanded first to anchor the distal section 934 and the proximal section 932 may be expanded later to anchor the proximal section 932 or vice versa.

The frame 902 may comprise a first end 916 (e.g., distal end) and/or a second end 918 (e.g., proximal end). The first end 916 and/or second end 918 may be at least partially flared and/or may increase in diameter relative to other portions of the frame 902.

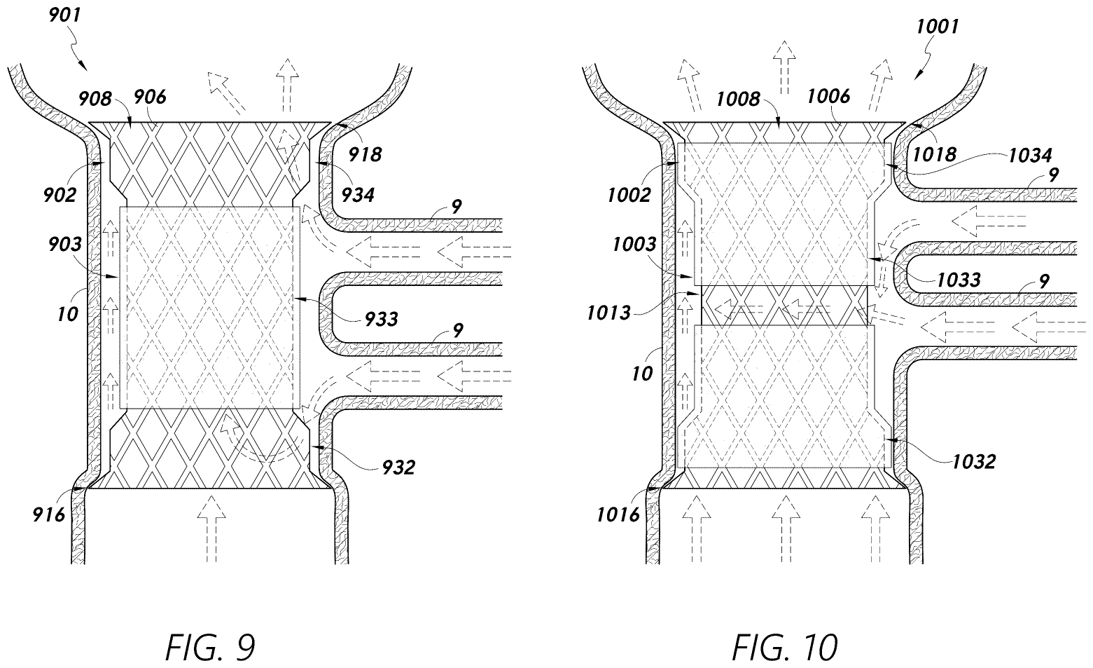

FIG. 10 illustrates another example stent 1001 configured to regulate blood flow within one or more blood vessels in accordance with one or more examples. The stent 1001 may comprise a generally collapsible frame 1002 attached to and/or enclosed by a covering 1003 composed of tissue, polymer, and/or similar materials. In some examples, the covering 1003 may be attached along an inner diameter and/or around an outer diameter of the frame 1002. The frame 1002 may comprise a network and/or mesh of struts 1006 extending around and/or forming one or more cells 1008 (e.g., diamond-shaped and/or other shaped openings through the frame 1002).

The frame 1002 may have a variable diameter. For example, the frame 1002 may comprise a midsection 1033 disposed between a proximal section 1032 and/or a distal section 1034. A diameter of the midsection 1033 may be less than diameters of the proximal section 1032 and/or distal section 1034. In some examples, the stent 1001 may be configured for placement such that the midsection 1033 may be disposed adjacent to one or more branching blood vessels (e.g., hepatic veins). Accordingly, the frame 1002 may be at least partially displaced from the branching blood vessels to allow blood flow from the blood vessels to escape into the IVC and/or other blood vessel in which the stent 1001 is disposed.

The midsection 1033 may have any suitable diameter. The diameter of the midsection 1033 may be determinative of an amount of blood flow allowed from the branching blood vessels. For example, if the diameter of the midsection 1033 is relatively small, the frame 1002 may allow a relatively high amount of blood flow from the branching blood vessels. Similarly, if the diameter of the midsection 1033 is relatively large, the frame 1002 may allow a relatively low amount of blood flow from the branching blood vessels.

In some examples, the diameter of the midsection 1033 may be at least partially adjustable prior to and/or following delivery of the stent 1001. For example, the stent 1001 may comprise one or more cords (e.g., sutures) and/or similar devices configured to function like a drawstring and/or similar function to cause a reduction and/or increase of the diameter of the midsection 1033. In some examples, one or more cords may be wound through the covering 1003 (e.g., through folds of the covering 1003) and/or through struts 1006 of the frame 1002 at or near the midsection 1033 and/or may be configured to be tightened and/or tensioned to cause a reduction in diameter of at least a portion of the covering 1003 and/or midsection 1033. The covering 1003 may be configured to form a seal around and/or within the frame 1002 to prevent blood flow from the branching blood vessels into a lumen formed by the stent 1001 and/or covering 1003.

The stent 1001 is shown disposed within an IVC 10 to regulate blood flow from one or more hepatic veins 10. However, the stent 1001 may be configured for use in other application and/or at other anatomies.

In some examples, the covering 1003 may comprise one or more holes (e.g., apertures, openings, bypass openings, etc.) configured to allow some blood flow through the covering 1003 and/or into the lumen formed by the covering 1003 and/or stent 1001. The one or more holes may be disposed at any portion(s) of the covering 1003 and/or may be disposed at a portion of the covering 1003 extending along the midsection 1033 of the stent 1001. In some examples, the holes may have variable shapes and/or sizes. Sizes and/or shapes of the holes may be determined based on a desired amount of blood flow reduction of the stent 1001. In some examples, the positioning and/or size of the holes may be adjusted prior to and/or following delivery of the stent 1001. For example, the covering 1003 may be flattened to cause the one or more holes to be exposed to the blood flow to increase an amount of blood flow through the covering 1003. The one or more holes may be oriented generally perpendicularly to the branching blood vessels and/or may be configured to allow blood flow from the branching blood vessels directly into the lumen.

The covering 1003 may be configured to extend at least partially along the proximal section 1032 and/or distal section 1034 of the frame 1002. In the example shown in FIGS. 10A and 10B, the covering 1003 extends along the proximal section 1032 and the distal section 1034. However, the covering 1003 may not extend along the distal section 1034 and/or the proximal section 1032. In some examples, the covering 1003 at the proximal section 1032 may be configured to prevent blood flow from branching blood vessels (e.g., hepatic veins 10) downwards and/or upstream of blood flow within the main blood vessel (e.g., IVC 10) and/or the covering 1003 at the distal section 1034 may be configured to prevent blood flow from the branching blood vessels upwards and/or downstream of blood flow within the main blood vessel.

In some examples, the covering 1003 may comprise a gap 1013 between a first section 1032 of the covering 1003 and a second section 1034 of the covering 1003. The gap 1013 may be disposed at the midsection 1033 of the frame 1002 and/or may be configured to allow increased blood flow from the branching blood vessels. In some examples, a size and/or position of the gap 1013 may be adjusted prior to and/or following delivery of the stent 1001. For example, on or more cords attached to the first section 1032 and/or second section 1034 may be configured to cause adjustment of the covering 1003 and/or gap 1013.

The stent 1001 may be at least partially balloon expandable and/or may be adjustable. For example, the stent 1001 may be expanded to a desired size for a particular application. The stent 1001 may be configured to maintain multiple desired forms.

In some examples, the proximal section 1032 and/or distal section 1034 may have increased diameters to facilitate anchoring of the stent 1001 within a blood vessel. Different portions of the frame 1002 may be expanded at different points to facilitate anchoring of the stent 1001. For example, the distal section 1034 may be expanded first to anchor the distal section 1034 and the proximal section 1032 may be expanded later to anchor the proximal section 1032 or vice versa.

The frame 1002 may comprise a first end 1016 (e.g., distal end) and/or a second end 1018 (e.g., proximal end). The first end 1016 and/or second end 1018 may be at least partially flared and/or may increase in diameter relative to other portions of the frame 1002.

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.
Example 1: A cardiac implant comprising a frame forming an inner lumen and configured for delivery into a blood vessel and a covering extending at least partially along the frame.
Example 2: The cardiac implant of any example herein, in particular example 1, wherein the frame has a tubular shape.
Example 3: The cardiac implant of any example herein, in particular example 1, wherein the frame comprises a network of one or more struts forming cells through the frame.
Example 4: The cardiac implant of any example herein, in particular example 3, wherein the frame comprises one or more posts extending along a length of the frame.
Example 5: The cardiac implant of any example herein, in particular example 4, wherein the one or more struts connect to the one or more posts.
Example 6: The cardiac implant of any example herein, in particular example 5, wherein the one or more struts comprise a series of wires extending circumferentially around the inner lumen.
Example 7: The cardiac implant of any example herein, in particular example 3, wherein the one or more struts have a zig-zag form.
Example 8: The cardiac implant of any example herein, in particular example 3, wherein the one or more struts form relatively large cells at a midsection of the frame and relatively small cells at or near a distal end or proximal end of the frame.
Example 9: The cardiac implant of any example herein, in particular example 3, wherein the cells are diamond-shaped.
Example 10: The cardiac implant of any example herein, in particular example 1, wherein the frame comprises a midsection having a smaller width than a distal section or proximal section of the frame.
Example 11: The cardiac implant of any example herein, in particular example 1, wherein the frame comprises a flared distal end or flared proximal end.
Example 12: The cardiac implant of any example herein, in particular example 1, wherein the covering has a first set of attachment points at or near a proximal end of the frame and a second set of attachment points at or near a distal end of the frame.
Example 13: The cardiac implant of any example herein, in particular example 12, wherein the second set of attachment points is approximately half the first set of attachment points.
Example 14: The cardiac implant of any example herein, in particular example 13, wherein the covering forms one or more folds at or near the distal end of the frame.
Example 15: The cardiac implant of any example herein, in particular example 14, wherein the one or more folds are in-line with attachment points at the proximal end of the frame and the covering has a tapered form between the one or more folds and the proximal end of the frame.
Example 16: The cardiac implant of any example herein, in particular example 14, further comprising a cord extending through the one or more folds and configured to cinch the covering together at or near the distal end of the frame.
Example 17: The cardiac implant of any example herein, in particular example 1, wherein the frame comprises two or more posts extending longitudinally along the frame, the covering attaches to each of the posts at or near a proximal end of the frame, and the covering attaches to approximately half of the posts at or near a distal end of the frame.
Example 18: The cardiac implant of any example herein, in particular example 17, wherein the covering attaches in an alternating manner to the half of the posts at or near the distal end of the frame.
Example 19: The cardiac implant of any example herein, in particular example 18, wherein the covering forms one or more folds at or near the distal end of the frame.
Example 20: The cardiac implant of any example herein, in particular example 19, wherein the one or more folds are in-line with attachment points at the proximal end of the frame and the covering has a tapered form between the one or more folds and the proximal end of the frame.
Example 21: The cardiac implant of any example herein, in particular example 19, further comprising a cord extending through the one or more folds and configured to cinch the covering together at or near the distal end of the frame.
Example 22: The cardiac implant of any example herein, in particular example 1, wherein the covering comprises one or more bypass holes to allow blood flow through the covering.
Example 23: The cardiac implant of any example herein, in particular example 1, wherein the covering comprises a proximal section and a distal section with a gap between the proximal section and the distal section.
Example 24: The cardiac implant of any example herein, in particular example 1, wherein the frame comprises a proximal section, a distal section, and a midsection between the proximal section and the distal section, and wherein the covering extends at least partially along the midsection.
Example 25: The cardiac implant of any example herein, in particular example 24, wherein the covering extends at least partially along the proximal section of the frame.
Example 26: The cardiac implant of any example herein, in particular example 25, wherein the covering does not extend along the distal section of the frame.
Example 27: The cardiac implant of any example herein, in particular example 1, wherein the covering is configured for placement adjacent to one or more branching blood vessels.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y, and at least one of Z to each be present.

It should be appreciated that in the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

Each element of each example and its respective elements disclosed herein can be used with any other example and its respective elements disclosed herein. All dimensions listed are by way of example, and devices according to the invention may have dimensions outside those specific values and ranges. The dimensions and shape of the device and its elements depend on the particular application. Unless otherwise noted, all technical and scientific terms used herein have the same meaning as commonly understood by one of ordinary skill in the art to which this disclosure belongs. In order to facilitate review of the various examples of the disclosure, the following explanation of terms is provided:

The singular terms “a”, “an”, and “the” include plural referents unless context clearly indicates otherwise. The term “or” refers to a single element of stated alternative elements or a combination of two or more elements, unless context clearly indicates otherwise. The term “includes” means “comprises.” For example, a device that includes or comprises A and B contains A and B, but may optionally contain C or other components other than A and B. Moreover, a device that includes or comprises A or B may contain A or B or A and B, and optionally one or more other components, such as C.

The term “subject” refers to both human and other animal subjects. In certain examples, the subject is a human or other mammal, such as a primate, cat, dog, cow, horse, rodent, sheep, goat, or pig. In a particular example, the subject is a human patient.

Although methods and materials similar or equivalent to those described herein can be used in the practice or testing of the present disclosure, suitable methods and materials are described below. In case of conflict, the present specification, including terms, will control. In addition, the materials, methods, and examples are illustrative only and not intended to be limiting.

In view of the many possible examples to which the principles of the disclosed invention may be applied, it should be recognized that the illustrated examples are only examples of the invention and should not be taken as limiting the scope of the invention. Rather, the scope of the invention is defined by the following claims. We therefore claim as our invention all that comes within the scope and spirit of these claims.

What is claimed is:

1. A cardiac implant for managing blood flow through a blood vessel, the cardiac implant comprising:
a frame comprising a network of struts forming one or more cells, the frame forming an inner lumen and sized for delivery into the blood vessel and being expandable to expand into contact with walls of the blood vessel; and
a covering extending at least partially along the frame and across at least some of the one or more cells to at least partially restrict lateral blood flow from one or more branching blood vessels into the blood vessel, at least a portion of the covering tapering away from the frame towards a distal end of the frame to form one or more folds at or near the distal end of the frame when the frame is expanded into contact with the walls of the blood vessel.

2. The cardiac implant of claim 1, wherein the covering extends along a full circumference of the frame.

3. The cardiac implant of claim 1, wherein the struts form relatively large cells at a midsection of the frame and relatively small cells at or near the distal end or a proximal end of the frame.

4. The cardiac implant of claim 1, wherein the frame comprises a midsection having a smaller width than a distal section or proximal section of the frame.

5. The cardiac implant of claim 1, wherein at least part of the distal end is flared.

6. The cardiac implant of claim 1, further comprising a cord extending through the one or more folds and configured to cinch the covering together at or near the distal end of the frame.

7. The cardiac implant of claim 1, wherein:
the frame comprises two or more posts extending longitudinally along the frame;
the covering attaches to each of the posts at or near a proximal end of the frame; and
the covering attaches to approximately half of the posts at or near the distal end of the frame.

8. The cardiac implant of claim 7, wherein the covering attaches in an alternating manner to the half of the posts at or near the distal end of the frame.

9. The cardiac implant of claim 8, wherein the one or more folds are in-line with attachment points at the proximal end of the frame.

10. The cardiac implant of claim 1, wherein the covering comprises one or more bypass holes to allow blood flow through the covering.

11. The cardiac implant of claim 1, wherein the covering comprises a proximal section and a distal section with a gap between the proximal section and the distal section.

12. The cardiac implant of claim 1, wherein the frame comprises a proximal section, a distal section, and a midsection between the proximal section and the distal section, and wherein the covering extends at least partially along the midsection and the proximal section, and wherein the covering does not extend along the distal section.

13. A cardiac implant for managing blood flow through a blood vessel, the cardiac implant comprising:
a frame comprising a network of struts forming one or more cells and a set of posts extending longitudinally along the frame, the frame forming an inner lumen and sized for delivery into the blood vessel and being expandable to expand into contact with walls of the blood vessel; and
a covering extending at least partially along the frame and across at least some of the one or more cells to at least partially restrict lateral blood flow from one or more branching blood vessels into the blood vessel, the covering being coupled to each post of the set of posts at a proximal end of the frame and coupling to a subset of posts of the set of posts at a distal end of the frame, the subset of posts being less than the set of posts.

14. The cardiac implant of claim 13, wherein the covering forms one or more folds at or near the distal end of the frame.

15. The cardiac implant of claim 13, wherein the struts form relatively large cells at a midsection of the frame and relatively small cells at or near a distal end or a proximal end of the frame.

16. The cardiac implant of claim 13, wherein
the covering attaches to approximately half of the posts of the set of posts at the distal end of the frame.

17. A cardiac implant for managing blood flow through a blood vessel, the cardiac implant comprising:
a frame comprising a network of struts forming one or more cells, the frame forming an inner lumen and sized for delivery into the blood vessel and being expandable to expand into contact with walls of the blood vessel, the frame comprising a midsection having a smaller width than a distal section or proximal section of the frame to create a gap between the midsection and the walls of the blood vessel; and
a covering extending at least partially along the midsection of the frame and across at least some of the one or more cells to at least partially restrict lateral blood flow from one or more branching blood vessels into the blood vessel, wherein the covering does not extend beyond the midsection of the frame.

18. The cardiac implant of claim 17, wherein the covering comprises one or more bypass holes to allow blood flow through the covering.

19. The cardiac implant of claim 17, wherein the covering comprises a proximal portion and a distal portion with a separation between the proximal portion and the distal portion.

20. The cardiac implant of claim 17, wherein the frame comprises one or more flared ends.
